(19) United States
(12) Patent Application Publication
(10) Pub. No.: US 2022/0405475 A1
(43) Pub. Date: Dec. 22, 2022

(54) SYSTEM AND METHOD FOR IMPROVED FEATURE DEFINITION USING SUBSEQUENCE CLASSIFICATION

(71) Applicant: WINTERLIGHT LABS INC., Toronto (CA)

(72) Inventors: Benjamin EYRE, Toronto (CA); Aparna BALAGOPALAN, Toronto (CA); Jekaterina NOVIKOVA, Toronto (CA)

(21) Appl. No.: 17/780,220

(22) PCT Filed: Dec. 11, 2020
(86) PCT No.: PCT/CA2020/051715

§ 371 Date: May 26, 2022

Related U.S. Application Data
(60) Provisional application No. 63/034,087, filed on Jun. 3, 2020.
(60) Provisional application No. 62/946,713, filed on Dec. 11, 2019.

(51) Int. Cl. G06F 40/284; G16H 50/70; G06N 5/02; G10L 15/18

(52) U.S. Cl. CPC G06F 40/284; G16H 50/70; G06N 5/022; G10L 15/18

(57) ABSTRACT

A feature set for performing classification of datasets such as speech transcripts by a machine learning classifier model is constructed using identification of features of interest through classification of subsequences of the dataset. An anchor comprising a class-differentiating token is identified, and subsequences of different lengths comprising the anchors and surrounding tokens are generated. The subsequence length producing a best performing classifier is selected. A feature set is then generated using transcript-level aggregates of token-level features for tokens in the dataset within that subsequence lengths length. The feature set may be added to a previously defined feature set for the dataset.

### CROSS-REFERENCE TO RELATED APPLICATIONS

This application claims priority to U.S. Provisional Application No. 62/946,713 filed Dec. 11, 2019 and to U.S. Provisional Application No. 63/034,087 filed Jun. 3, 2020, the entireties of which are incorporated herein by reference.

### TECHNICAL FIELD

The present disclosure relates to development of feature sets for machine learning models for speech classification-related tasks, and further to improvements in feature engineering for speech classification-related tasks and in machine-learning based detection of cognitive impairment.

### TECHNICAL BACKGROUND

Word and sentence embedding-based machine learning methods have been shown to be successful for natural language processing tasks. These approaches stand as an alternative to classical feature engineering approaches, where carefully crafted features, such as word length or part of speech tag, are extracted from text and used as input. Nevertheless, the use of more engineered features remains common in fields where ease of interpretability is important, such as health care, and particularly in detection and diagnosis of cognitive impairment.

However, manual feature engineering is time and resource consuming. Further, despite the fact that manual feature selection is based on domain knowledge, it can potentially result in features that do not enhance model performance.

### BRIEF DESCRIPTION OF THE DRAWINGS

In drawings which illustrate by way of example only embodiments of the present application,

FIG. 1 is a flowchart illustrating a process according to the examples and embodiments herein.

FIG. 2 is a schematic diagram depicting the relationship between contexts and distances between tokens and anchors in accordance with an example described below.

FIG. 3 is a table setting out parameters of the best performing sequential classifier machine learning models in a subsequence classifying step.

FIG. 4 is a table setting out parameters of the best performing models for each feature set in a transcript classifying step.

FIG. 5 is a table setting out transcript classification performance for each feature set's best performing classification model.

FIG. 6 is a schematic illustrating a networked environment suitable for implementing the embodiments and examples described herein.

### DETAILED DESCRIPTION

While successes have been reported for embedding-based machine learning (ML) methods on natural language processing (NLP) tasks, engineered features continue to be important in some fields. For example, in the health care field it may be preferable to rely on engineered features to facilitate interpretation of a model; the correlation between engineered features and a classification or prediction result is more easily interpreted and explainable by or to clinicians. Thus, feature engineering remains an important practice in health care-related tasks, including detection of cognitive impairment (CI). Furthermore, the performance of embedding-based ML methods is adversely impacted by the use of smaller data sets. This is an additional concern in the health care field. For example, currently available datasets for speech-based cognitive impairment detection are generally limited and noisier as compared to the (healthy-speech) language corpora available for more general NLP tasks such as translation or sentiment analysis. However, manual feature engineering consumes time and resources. Further, while domain knowledge may assist the selection of some useful features, domain knowledge may also prove distracting in that some choices for feature extraction may not, in fact, enhance model performance.

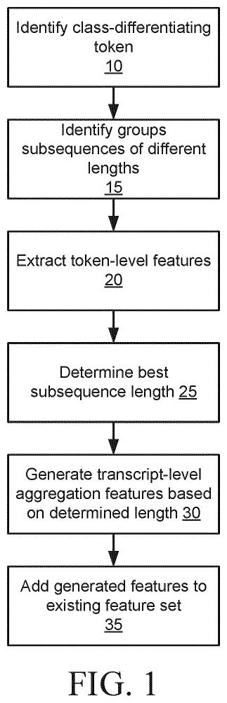

Thus, in accordance with the experiments and examples described herein, a new approach to feature engineering is provided that leverages sequential machine learning models and domain knowledge to predict which features are likely to help performance of a classifier model, for example for the purpose of diagnosis. Briefly, referring to FIG. 1, a token of interest, referred to as an “anchor”, is identified 10; subsequences of varying length centered around the anchor, are identified from each entry in a data set, such as transcripts. The subsequences are grouped into subsets based on maximum length 15. Token-level features are extracted for each of the tokens in the subsequence, to be used as input to a sequential classifier machine learning model 20. Such token-level features and the anchor may be chosen based on in-domain knowledge. Next, a best subsequence length is determined 25, based on which subsequence length results in the best performing classification. For example, a sequential machine learning model may be cross-validated on each of the subsequence data subsets in a subsequence classification exercise. From these results, an indication of how much distinguishing information can be extracted from tokens within a specified range of the anchor can be determined. The best performing sequential classifier determines the best length. The best performing classifier may be considered to be the classifier with the highest accuracy, as set out in the example here. However, the best performing classifier may be determined according to other metrics, such as meeting another target accuracy level (e.g., a threshold value), or the classifier with the highest precision, sensitivity, etc. or meeting a threshold value for one or more metrics. Once the maximum length is determined, transcript-level aggregations of the token-level features of the tokens within that maximum length or distance of any anchors in the transcript are generated 30, as explained below. These newly engineered features may be added to an existing feature set 35 for the entry or transcript to effectively increase the signal to noise ratio of the existing feature set, since the transcript-level aggregations of the token-level features add focus to or amplify characteristics of the transcript that may be more discriminating in a classification exercise.

Concrete examples of this are set out below, based on a standard dataset of CI speech. These examples utilize the Dementiabank dataset described below, and select a pause in speech as the anchor, as motivated by previous studies suggesting that various characteristics of the words following a pause could be an indicator for CI (Calley et al. 2010. Subjective report of word-finding and memory deficits in normal aging and dementia. Cognitive and Behavioral Neurology: Official Journal of the Society for Behavioral and Cognitive Neurology, 23(3):185; Mack et al. 2013. Word-finding pauses in primary progressive aphasia (PPA): Effects of lexical category; Seifart et al. 2018. Nouns slow down speech across structurally and culturally diverse languages. Proceedings of the National Academy of Sciences, 115(22):5720-5725). These examples demonstrate that CI classification accuracy improves by 2.3% over a strong baseline when using features produced by this new feature engineering method, and that moreover, the use of features from the two tokens both preceding and succeeding a pause could enhance transcript-level classification performance when diagnosing CI. Further, the examples below demonstrate how this method can be used to assist classification in fields where interpretability is important, such as health care.

### Example Data Sources

Data subsets were obtained from Dementiabank (DB), a large public dataset of pathological speech, available at dementia.talkbank.org (Becker et al., 1994). DB contains audio files and corresponding transcripts of participants describing the “Cookie Theft” image. Out of the 286 participants, 193 were diagnosed with some form of CI (N=321 transcripts), and 93 were healthy controls (HC; N=229 transcripts).

### Baseline Reference

To provide a baseline for comparison, an initial set of 509 linguistic and acoustic features were extracted from each transcript. This feature set is referred to here as an “original” feature set. Further, feature selection was performed on the original feature set to find the k=85 features leading to the greatest performance. These original and top 85 feature sets were used to provide baselines to benchmark transcript-level classification performance. Additionally, as will be seen below, the original feature set was extended with newly-engineered features.

The linguistic and acoustic features (Fraser et al. Linguistic features identify Alzheimer's disease in narrative speech. Journal of Alzheimer's Disease, 49(2):407-422; Toth et al. A speech recognition-based solution for the automatic detection of mild cognitive impairment from spontaneous speech. Current Alzheimer Research, 15(2):130-138) come from one of the following eight categories:
Information Units: Semantic measures, pertaining to the ability to describe concepts and objects in the picture.
Discourse Mapping: Features that help identify cohesion in speech using a visual representation of message organization in speech. Each word is represented as a node to build a ‘speech graph’ (Mota et al. 2012. Speech graphs provide a quantitative measure of thought disorder in psychosis. PloS One, 7(4):e34928), for the whole transcript. Examples of features then extracted include number of edges in this graph, number of self-loops, etc.
Coherence: Semantic continuity that listeners perceive between utterances (locally or globally).
Lexical Complexity and Richness: Different measures of lexical qualities and variation. Examples of features include average age of acquisition, and number of occurrence of various POS tags.
Sentiment: Sentiment lexical norms from Warriner et al., 2013 (Warriner et al. 2013. Norms of valence, arousal, and dominance for 13,915 English lemmas. Behavior Research Methods, 45(4):1191-1207). Examples include average sentiment valence over verbs, and average sentiment dominance over nouns.
Syntactic Complexity: Different measures to analyze the syntactic complexity of speech including features including number of occurrence of various production rules and mean length of clause (in words)
Word finding Difficulty: Features quantifying difficulty in finding the right words, including various pause features such as number of filled pauses, and pause word ratio.
Acoustic: Voice markers such as MFCC coefficients and Zero Crossing Rate (ZCR) related features.

### Subsequence Data Subsets

To conduct subsequence classification, subsequences of varying length were extracted from each transcript. For each transcript in DB, each utterance containing the identified anchor, in this case a pause, was extracted and labelled as positive if the sample contained CI speech, or negative otherwise (labelled as HC).

Three data subsets were created by extracting subsequences of at most one, two, or three tokens around (before and after) the anchor. These are referred to as Context 1 (DB-C1), Context 2 (DB-C2), and Context 3 (DB-C3), respectively. If there were less than two or three tokens before or after an anchor in an utterance, the largest possible sequence of tokens was extracted. In addition, one data subset was created including full utterances that included the anchor, DB-Utt. Identical subsequences found in both classes (HC and CI) were removed. Additionally, if multiple identical subsequences were found in only one of the classes, all but one of them were removed. A breakdown of the subsequences (after deduplication) is set out in Table 1, below.

TABLE 1
Overview of the number of samples (subsequences
or transcripts) in different subsets of DB.

| Data Subset | HC | CI | | Total |
|---|---|---|---|---|
| DB (transcripts) | 229 (42%) | 321 | (58%) | 550 |
| DB-C1 | 317 (33%) | 645 | (67%) | 962 |
| DB-C2 | 511 (35%) | 963 | (65%) | 1,474 |
| DB-C3 | 529 (35%) | 980 | (65%) | 1,509 |
| DB-Utt | 755 (42%) | 1,059 | (58%) | 1,814 |

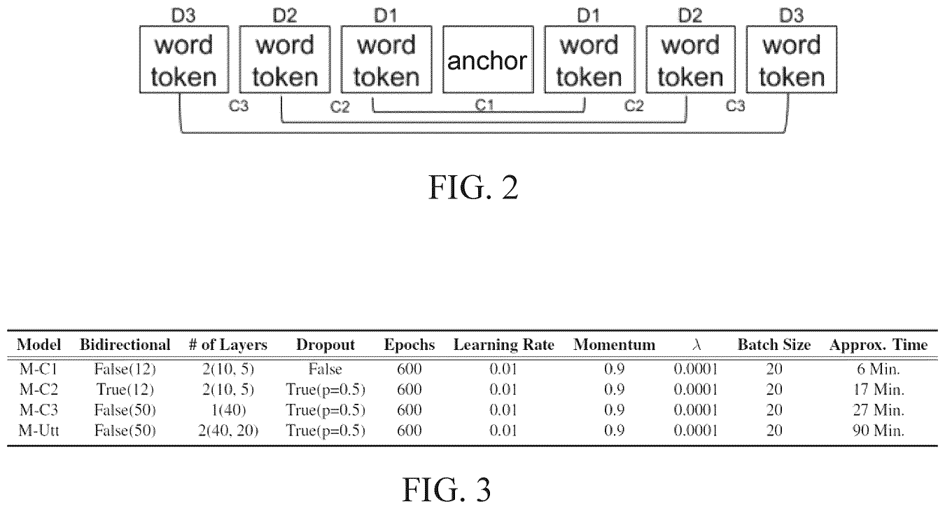

Tokens that are next to the pause are referred to as Distance 1 (D1); tokens that are one token away from the pause as Distance 2 (D2); and tokens that are two tokens away from the pause as Distance 3 (D3). The differences between context and distance are illustrated in FIG. 2, where context sizes (C1, D2, C3) correlate to the maximum distance of a member word token from the anchor (D1, D2, D3).

### Subsequence Feature Extraction

For the purpose of subsequence classification, features were extracted at the token level for each of the subsequence data subsets, i.e., for each token in a subsequence. The features selected for extraction at this step were those with a clear analogue in the original feature set. For instance, a transcript-level feature of average word length has a clear token-level analogue of individual word length. Thus, one example of an analogous feature at the transcript and token levels is a case where the transcript-level feature is a transcript mean, aggregate, or other composite representation of the token-level feature.

The extracted features may be categorized as follows:
Word length: Length of the word, both in syllables and letters.
Sentiment: Three measures of the type and intensity of reaction a word produces. (Warriner et al., 2013)
Concreteness: Measure of the degree to which a word refers to a perceptible entity. (Brysbaert et al. 2014. Concreteness ratings for 40 thousand generally known English word lemmas. Behavior Research Methods, 46(3):904-911.)
Imageability: How easy it is for a word to elicit a mental image. (Stadthagen-Gonzalez et al. 2006. The Bristol norms for age of acquisition, imageability, and familiarity. Behavior Research Methods, 38(4):598-605.)
Age of acquisition: Average age that the word is learned. (Kuperman et al. 2012. The 385+ million word corpus of contemporary American English (1990-2008+). Behavior Research Methods, 44(4):978-990.)
Frequency: Word counts in a corpus of over 385 million words. (Davies, 2009. Age-of-acquisition ratings for 30,000 English words. International Journal of Corpus Linguistics, 14(2):159-190.)
Familiarity: The perceived popularity of a word.(Stadthagen-Gonzalez et al., 2006)
Part of speech: Grammatical category of the word, based on parts of speech categories defined in the spaCy library (available online at spacy.io): ‘ADJ’ (adjective), ‘ADP’ (adposition), ‘ADV’ (adverb), ‘AUX’ (auxiliary), ‘CONJ’ (conjunction), ‘CCONJ’ (coordinating conjunction), ‘DET’ (determiner), ‘INTJ’ (interjection), ‘NOUN’ (noun), ‘NUM’ (numeral), ‘PART’ (particle), ‘PRON’ (pronoun), ‘PROPN’ (proper noun), ‘PUNCT’ (punctuation), ‘SCONJ’ (subordinating conjunction), ‘SYM’ (symbol), ‘VERB’ (verb), ‘X’ (other), ‘SPACE’ (space).

To ensure that each anchor (pause in this example) had at least one token preceding and following it, start and end tokens were added to each utterance in DB in DB-Utt. For those tokens that did not have the features identified above (such as start, end, and anchor tokens), those features were given a value of zero with the exception of a unique part of speech value to distinguish these tokens. The sentiment, concreteness, imageability, age of acquisition, frequency, and familiarity features were extracted for the tokens themselves, as well as for the lemmatized token (i.e., a total of two features of each type except for sentiment, for which there were six in total). Feature values for the two word length features or the part of speech feature were not obtained for the lemmatized token. The part of speech feature was generated as a five-dimensional, randomly initialized embedding, thus producing a total of 23 dimensions for each token.

Any missing values for word tokens were imputed with feature means, which were normalized with respect to the features means and standard deviations of the word tokens (extending anchors and start/end tokens). Consequently, each subsequence in the DB-C1, DB-C2, DB-C3, and DB-Utt subsets were represented by a T×23 matrix, where T is the length of the subsequence in tokens (ignoring the anchor). Thus, for example, T=4 for a subsequence in DB-C2.

### Subsequence Classification

Subsequence classification was carried out using sequential machine learning models. Gated Recurrent Unit (GRU)-based models with attention (Cho et al., 2014; Yang et al., 2016), were used, with model parameters turned for each of the data subsets. Each model consisted of a GRU that took the subsequence representations as input, and output to a feed forward neural network which then made predictions. The attention mechanism used a linear layer with as many hidden units as the GRU, as well as a context vector with as many dimensions as the GRU has hidden units. An extensive search was conducted in terms of finding the most effective model parameters for each data subset. Each model was tested with variations on the number of intermediate layers in the predicting feed-forward network (1, 2 or 3), the addition of dropout, whether the GRU was bidirectional, and the number of hidden units in each layer (large or small, where large has approximately 4 times as many hidden units in each layer as small). This created a total of 24 trials per data subset. All the models were created with Pytorch (Paszke et al., 2017. Pytorch: Tensors and dynamic neural networks in Python with strong GPU acceleration. PyTorch: Tensors and dynamic neural networks in Python with strong GPU acceleration, 6). Each model was trained for 600 epochs using SGD as an optimizer, learning rate=0.01, momentum=0.9, L2 regularization with λ=0.0001, batch size of 20, a Cosine Annealing learning rate scheduler, and cross entropy loss. Each layer with the exception of the final layer used the rectified linear unit (ReLU) activation function. Additionally, training scripts were run using the central processing unit of a p2.xlarge Elastic Compute Cloud instance provided by Amazon Web Services (aws.amazon.com/ec2/).

Five-fold cross validation was performed with each of the DB-C1, DB-C2, DB-C3, and DB-Utt data subsets, and the best performing model was selected for DB-C1, DB-C2, DB-C3, and DB-Utt. A model was only considered if it was able to meet or exceed the specificity achieved by a model that was once state of the art, 28.8% (Di Palo et al. 2019. Enriching neural models with targeted features for dementia detection. arXiv preprint arXiv:1906.05483).

A summary of the parameters used for each of the best performing models is set out in FIG. 3, with the approximate time required to complete cross-validation. The number of hidden units in the GRU is indicated in the column indicating whether the GRU was bidrectional, and the number of hidden units in each layer of the predicting network is indicated next to the column indicating the number of layers. Model M-C1 (the best performing model for DB-C1) obtained an accuracy (percentage) of 59.6±2.6; M-C2 (the best performing model for DB-C2) obtained an accuracy of 60.7±2.5; M-C3 (DB-C3), 59.8±0.9; and M-Utt (DB-Utt), 60.3±1.0. Accuracy was averaged across four random seeds.

### New Transcript-Level Feature Extraction

The results of the subsequence classification informed the extraction of new transcript-level aggregate features from the DB transcripts in order to emphasize the context of interest, that is to say the context that appeared to be the most useful. It was theorized that aggregate features need only be extracted for those tokens in the transcript that were within the distance that produced the greatest cross-validated accuracy during subsequence classification, as the subsequence classification performance indicates that features found in that range would be the most distinguishing. Based on the results above, the highest accuracy was obtained with DB-C2. Therefore, these new aggregate features should only need to be extracted for tokens found at the D1 and D2 positions with reference to anchors in the transcript, and not for those tokens in the D3 position.

To validate this, five transcript-level feature sets for all three contexts were created:

features aggregated from tokens at the D1 position in reference to a pause (F-D1);

features aggregated from the D2 position (F-D2);

features aggregated from the D3 position (F-D3);

the combination of F-D1, F-D2, and F-D3 (F-C3); and

the combination of F-D1 and F-D2 (F-C2).

The new aggregate features were aggregations of token-level features of those tokens at the D1, D2, or D3 position in a transcript. Aggregations were generated using methods appropriate to the feature type. For instance, a continuous feature such as word length could be aggregated by simply taking an average of the feature of each of the tokens within the contexts of interest in a transcript. An example of this would be the aggregation of the token-level feature of word length of tokens at the D2 location: such an aggregation could be the mean of the word length of all word tokens at D2 relative to each of the anchors in a given transcript. Such aggregates were taken for the continuous token-level features previously extracted for the subsequences: word length, sentiment, concreteness, imageability, age of acquisition, frequency, and familiarity. Discrete or categorical features may be aggregated using a count (preferably normalized) or a ratio of the feature of each of the tokens within all contexts of interest in a transcript. Thus, for example, the aggregation of the token-level part-of-speech feature was computed by taking the total number of times that part of speech occurred at a specified distance from the anchor, divided by the total number of anchors occurring in the transcript, and multiplied by the percent of words in the transcript that were parts of speech. (As the anchor in this example is a pause, they were not considered when calculating mean feature values.)

It will be appreciated from the above examples that the new aggregate features, while computed as a feature for a transcript, are aggregates determined from the tokens within the context of interest (tokens within D1, D2, or D3, depending on the context that produced the greatest cross-validated accuracy during subsequence classification). Thus, while they are transcript-level features (features of a transcript), they are transcript-level aggregations of token-level features for tokens within the context of interest. By contrast, a typical transcript-level feature such as those in the original feature set are simple aggregates, such as the mean of the word length of all word tokens in a transcript irrespective of their distance from an anchor.

### Transcript Classification

The efficacy of the above feature engineering approach was evaluated by classifying DB transcripts with the assistance of the newly-engineered features described above. 10-fold cross-validation was performed with a variety of feature sets, using Random Forest (100 trees), Gradient Boosting Estimator (with 150 estimators), Support Vector Machine (SVM) with Radial Basis Function (RBF) kernel, and 2-layer neural network (NN, 10 units, Adam optimizer, 200 epochs with learning rate initialized to 0.01) classifiers, in addition to an ensemble of all four of the aforementioned classifiers (Ens) (Pedregosa et al., 2011. Scikit-learn: Machine learning in Python. The Journal of Machine Learning Research, 12:2825-2830). Classification of the DB transcripts used the two baselines described above, namely the original feature set and the top 85 feature set.

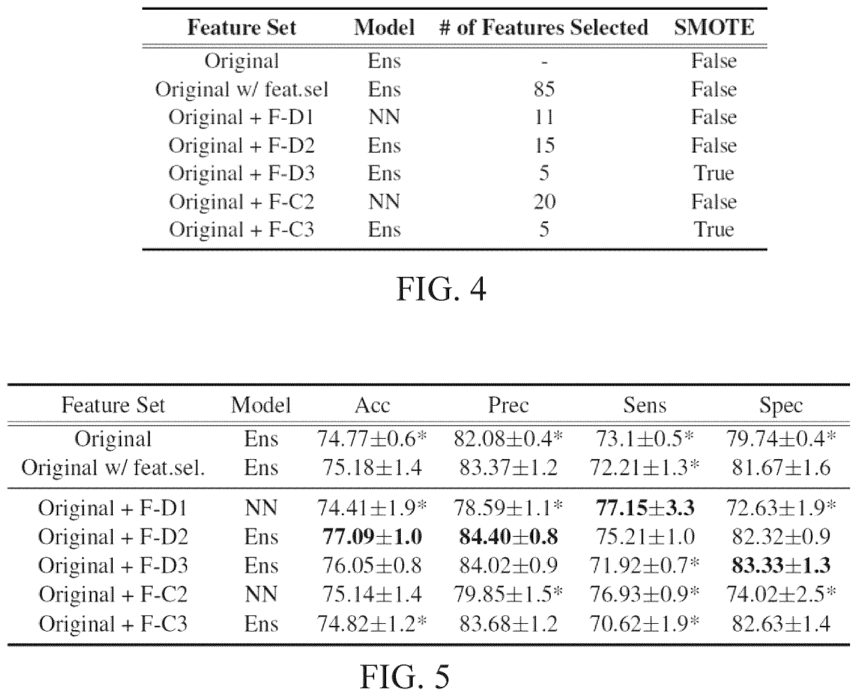

Additionally, the original feature set was extended with the k best features from each of the newly-generated feature sets (the transcript-level aggregations of token-level features described above), separately. The k best features separately determined for each extended feature set, (feature selection with k=3, 5, 7, 9, 11, 13, 15, 20, 25, 30, and all features), jointly with whether or not Synthetic Minority Over-sampling Technique (SMOTE) was used (Chawla et al., 2002. Smote: synthetic minority over-sampling technique. Journal of Artificial Intelligence Research, 16:321-357), and the model used. For feature selection on the original feature set, feature selection with k=20, 25, 30, 35, 40, 45, 50, 55, 60, 65, 70, 75, 80, 85, 90, 95, 100, 150, 200, 250, 300, and 350 was attempted. The best performing configuration for each extended feature set is set out in FIG. 4.

FIG. 5 reports the accuracy (Acc), precision (Prec), sensitivity (Sens), and specificity (Spec) for the classification model that achieved the greatest cross-validated accuracy for each feature set, separately. Bold figures indicate the best performance, and * indicates significance (p<0.05) when compared to the model using F-D2 features. The original feature sets extended by the F-D1, F-D2, and F-C2 sets incorporate transcript-level aggregations of token-level features for tokens within D2 of the anchors in the transcript. As can be seen in the results of FIG. 5, the highest transcript classification accuracy, 77.09%±1.0, was achieved by an ensemble model that used the original feature set extended by F-D2. Using one of the four random seeds used to produce these results, the model using the original feature set extended by F-D2 was able to achieve an accuracy of 78.36%, the same as the single-seed state of the art accuracy of 78%, based entirely on domain knowledge-based manual (not automated) choice of features (Hernandez-Dominguez et al., 2018. Computer-based evaluation of Alzheimer's disease and mild cognitive impairment patients during a picture description task. Alzheimer's & Dementia: Diagnosis, Assessment & Disease Monitoring, 10:260-268).

It can be seen from the foregoing examples that features from tokens within two tokens of the anchor—the pause in these examples—were the most effective in enhancing both subsequence and transcript classification. To determine how a statistical analysis was conducted on the token-level and transcript-level features. Two-sided t-tests between features extracted from tokens found at D1, D2, and D3 from different classes showed similar patterns for features that were significantly different between classes for both the token and transcript-level. Larger concentrations of distinguishing features were found at D1 and D2 than at D3, as can be seen in Table 2, below. Without wishing to be bound by theory, this may explain why features from the D2 position are so effective when used in both tasks.

TABLE 2
Number of features that are significantly different between
classes according to two-sided t-tests for each distance.

| Distance | Token-Level | Transcript-Level |
|---|---|---|
| D1 | 18 | 12 |
| D2 | 21 | 12 |
| D3 | 7 | 6 |

It may also be seen from the results that extending the original feature set with F-D3 features on their own may be more effective than extending with F-D1 features on their own, as both the extended F-D3 and F-C3 feature sets produced greater transcript-level accuracy than the extended F-D1 set (FIG. 5). This resembles the results of the best performing subsequence classification models. Thus, subsequence classification may be able to provide better insight into potential transcript classification performance than traditional statistical testing.

The foregoing results thus demonstrate the efficacy of the novel feature engineering technique. Further, the results also indicate that in the context of detecting and diagnosing CI, tokens taken both before and after a pause anchor are helpful, and that contrary to prior clinical research, the token immediately following a pause is not necessarily as helpful; as can be seen above, Context 1 was not the best performing context in these examples.

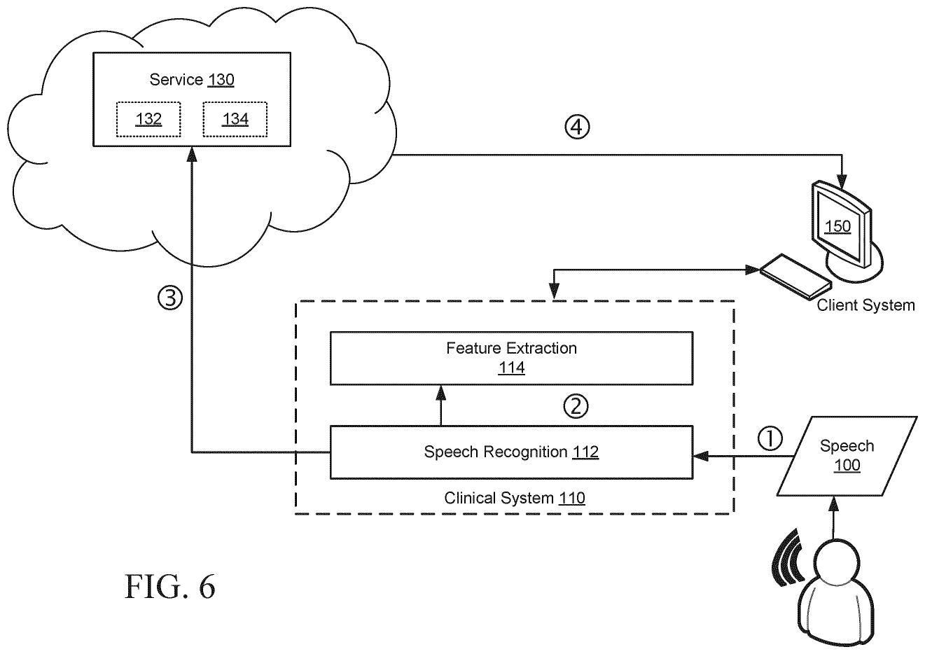

The examples and embodiments described above may be advantageously implemented in a data processing system, such as the example system depicted in FIG. 6. In the example of FIG. 6, the system is directed to detection of impaired speech, and optionally may be carried out using a cloud-based or otherwise remote analysis service 130 communicating with one or more client systems 150 over a network. Such a remote service 130 is preferably operated in compliance with any applicable privacy legislation, such as the United States Health Insurance Portability and Accountability Act of 1996 (HIPAA).

Individual client systems 150 may be computer systems in clinical or consultation settings, communicating with the remote service 130 over a wide area network (e.g., the Internet). It is contemplated that users may implement clinic or patient management software locally, and that preferably, personally identifying information (PII) of a patient—which can include recorded utterances—is stored securely, and even locally, to avoid accidental dissemination of PII by third-party services. This is reflected in the example data processing system of FIG. 6, in which a patient's speech is received and recorded 100 using any appropriate recording technology, and provided to a clinical system 110. The clinical system 110 may comprise the clinic or patient management software, or a dedicated application that communicates with the remote analysis service 130. The clinical system 110 may comprise a further remote server system or cloud-based system, accessible by the client system 150 via a network. The clinical system 110 may be operated or hosted by a third party provider, but in some implementations, it may be hosted locally in the clinical setting, e.g., co-located with the client system 150.

The clinical system 110 in this example is configured to receive the recorded speech (1), convert the recorded speech to text using a suitable speech recognition module 112 as would be known to those skilled in the art, and to provide the recognized speech (2) to a feature extraction module 114 which is configured to recognize the linguistic features of interest for use in classification, and to generate the feature set that will be used as input to a ML classifier system. In some implementations, a transcript of the subject's speech may be produced manually, and the featurization may be carried out manually as well. The generated feature set is then provided (3) to the remote analysis service 130; this feature set data may be provided to the remote analysis service 130 anonymously, for example identified only using a patient identification number.

The remote analysis service 130 may include modules for executing the classification model to perform detection/diagnosis 132 and to update/retrain the model 134. Of course, actual training/retraining of the model may be carried out outside the illustrated data processing system, with model artefacts imported into the remote analysis service 130 for execution in module 132. The remote analysis service 130 inputs the received feature set to the classification model 132 generate a classification result, which is then transmitted (4) over the network to the client system 150. The classification result may be an indication whether the received feature set is classified as CI or non-CI speech.

Thus, in accordance with the examples described above, there is provided a method for generating a feature set for a plurality of entries to be classified by a classification model according to a classification, the method comprising: identifying an anchor comprising a class-differentiating token in a data set comprising a plurality of entries to be classified according to a classification; for anchors found in each entry of the plurality of entries, identifying a plurality of subsequences of different lengths, each subsequence comprising a set of tokens around the anchors; determining which length of subsequence provides a target level of accuracy for the classification, the length defining a distance from an anchor; defining and storing a first feature set, the first feature set comprising transcript-level aggregates of token-level features for tokens located within the defined distance of anchors in the entries of the plurality of entries.

There is also provided a method of augmenting a previously-defined feature set for classifying a plurality of entries of a data set according to a classification, the method comprising identifying an anchor comprising a class-differentiating token in a data set comprising a plurality of entries to be classified according to a classification; for anchors found in each entry of the plurality of entries, identifying a plurality of subsequences of different lengths, each subsequence comprising a set of tokens around the anchors; determining which length of subsequence provides a target level of accuracy for the classification, the length defining a distance from an anchor; defining and storing a first feature set, the first feature set comprising transcript-level aggregates of token-level features for tokens located within the defined distance of anchors in the entries of the plurality of entries, and adding the first feature set to the previously-defined feature set to provide an extended feature set.

There is further provided a method of classifying a speech transcript, comprising: identifying at least one anchor comprising a class-differentiating token in the transcript; extracting features from the speech transcript according to a feature set, the feature set including a plurality of transcript-level aggregates of token-level features for tokens located within a defined distance of each of the at least one anchor; and providing the extracted features as input to a trained classifier model to obtain a classification.

There is also provided a method of diagnosing cognitive impairment from a speech transcript, the method comprising: obtaining a transcript of a subject's speech; identifying at least one anchor comprising pause in the speech transcript; extracting features from the speech transcript according to a feature set, the feature set including a plurality of transcript-level aggregates of token-level features for tokens located within a defined distance both before and after the at least one anchor; and providing the extracted features as input to a trained classifier model to obtain a classification of the speech transcript as indicative of cognitive impairment or not indicative of cognitive impairment.

There is further provided systems for implementing the above methods, for example comprising one or more processors in communication with suitable memory devices configured to implement the methods described herein. These systems may or may not be distributed or cloud-based networked systems.

The examples and embodiments are presented only by way of example and are not meant to limit the scope of the subject matter described herein. Individual features of each example or embodiment presented above may be combined, in whole or in part, with individual features of other examples or embodiments. Some steps or acts in a process or method may be reordered or omitted, and features and aspects described in respect of one embodiment may be incorporated into other described embodiments. Variations of these examples will be apparent to those in the art and are considered to be within the scope of the subject matter described herein. For example, depending on the nature of the classification task, longer contexts and subsequences may be selected for subsequence classification to identify larger contexts of interest than those discussed above. Different metrics may be used to select the best performing subsequence classifier, and thus the context length, as may be informed by the data (e.g., in an imbalanced data set, accuracy may not be used to determine the best-performing classifier).

The data employed by the systems, devices, and methods described herein may be stored in one or more data stores. The data stores can be of many different types of storage devices and programming constructs, including but not limited to RAM, ROM, flash memory, programming data structures, programming variables, and so forth. Code adapted to provide the systems and methods described above may be provided on many different types of computer-readable media including but not limited to computer storage mechanisms (e.g., CD-ROM, diskette, RAM, flash memory, computer hard drive, etc.) that contain instructions for use in execution by one or more processors to perform the operations described herein. The media on which the code may be provided is generally considered to be non-transitory or physical.

Computer components, software modules, engines, functions, and data structures may be connected directly or indirectly to each other in order to allow the flow of data needed for their operations. The data processing and computer systems described above may be provided in a single location, or may be distributed in a cloud environment, using techniques known to those skilled in the art. Those skilled in the art will know how to implement the example systems and methods described above. Various functional units have been expressly or implicitly described as modules, engines, or similar terminology, in order to more particularly emphasize their independent implementation and operation. Such units may be implemented in a unit of code, a subroutine unit, object, applet, script or other form of code. Such units may also be implemented in hardware circuits comprising custom circuits or gate arrays; field-programmable gate arrays; programmable array logic; programmable logic devices; commercially available logic chips, transistors, and other such components. As will be appreciated by those skilled in the art, where appropriate, functional units need not be physically located together, but may reside in different locations, such as over several electronic devices or memory devices, capable of being logically joined for execution. Functional units may also be implemented as combinations of software and hardware, such as a processor operating on a set of operational data or instructions.

Use of any particular term should not be construed as limiting the scope or requiring experimentation to implement the claimed subject matter or embodiments described herein. Any suggestion of substitutability of the data processing or computer systems or environments for other implementation means should not be construed as an admission that the invention(s) described herein are abstract, or that the data processing systems or their components are non-essential to the invention(s) described herein.

A portion of the disclosure of this patent document contains material which is or may be subject to one or more of copyright, design, or trade dress protection, whether registered or unregistered. The rightsholder has no objection to the reproduction of any such material as portrayed herein through facsimile reproduction of this disclosure as it appears in the Patent Office records, but otherwise reserves all rights whatsoever.

1-28. (canceled)

29. A method of classifying a speech transcript, comprising:
identifying at least one anchor comprising a class-differentiating token in the transcript;
extracting features from the speech transcript according to a defined feature set, the defined feature set including a plurality of transcript-level aggregates of token-level features for tokens located within a defined distance of each of the at least one anchor; and
providing the extracted features as input to a trained classifier model to obtain a classification.

30. The method of claim 29, wherein the tokens are located within a defined distance both before and after the at least one anchor.

31. The method of claim 29, wherein the classification is a classification of the speech transcript as indicative of cognitive impairment or not indicative of cognitive impairment.

32. The method of claim 29, wherein the at least one anchor comprises a pause in the transcript, and wherein the classification comprises a classification of the speech transcript as indicative of cognitive impairment or not indicative of cognitive impairment.

33. The method of claim 29, further comprising:
identifying the anchor in a data set comprising a plurality of entries to be classified according to a classification;
for anchors found in each entry of the plurality of entries, identifying a plurality of subsequences of different lengths, each subsequence comprising a set of tokens around the anchors;
determining which length of subsequence provides a best performing classification, the length defining a distance from an anchor, the distance comprising a number of tokens before and after the anchor; and
defining and storing a set of features comprising transcript-level aggregates of token-level features for tokens located within the defined distance of anchors in the entries of the plurality of entries.

34. The method of claim 33, wherein the transcript-level aggregates comprise at least one of a count or ratio of the token-level feature for the tokens located within the defined distance of the anchors in the entry, or an average of the token-level feature for the tokens located within the defined distance of the anchors in the entry.

35. The method of claim 33, wherein the set of features augments a previously-defined feature set to provide the defined feature set.

36. The method of claim 33, further comprising:
extracting and saving values for the extended feature set for each entry of the plurality of entries to provide a set of representations; and
training a classifier machine learning model using the set of representations to provide the trained classifier model.

37. The method of claim 29, further comprising obtaining the speech transcript by either transcribing a subject's recorded speech or by executing automated speech-to-text recognition on a subject's speech.

38. Non-transitory computer-readable media storing code which, when executed by one or more processors of a computer system, causes the computer system to implement:
identifying at least one anchor comprising a class-differentiating token in the transcript;
extracting features from the speech transcript according to a defined feature set, the defined feature set including a plurality of transcript-level aggregates of token-level features for tokens located within a defined distance of each of the at least one anchor; and
providing the extracted features as input to a trained classifier model to obtain a classification.

39. The non-transitory computer-readable media of claim 38, wherein the classification is a classification of the speech transcript as indicative of cognitive impairment or not indicative of cognitive impairment.

40. The non-transitory computer-readable media of claim 38, wherein the at least one anchor comprises a pause in the transcript, and wherein the classification comprises a classification of the speech transcript as indicative of cognitive impairment or not indicative of cognitive impairment.

41. The non-transitory computer-readable media of claim 38, wherein the computer system is further caused to implement:
identifying the anchor in a data set comprising a plurality of entries to be classified according to a classification;
for anchors found in each entry of the plurality of entries, identifying a plurality of subsequences of different lengths, each subsequence comprising a set of tokens around the anchors;
determining which length of subsequence provides a best performing classification, the length defining a distance from an anchor, the distance comprising a number of tokens before and after the anchor; and
defining and storing a set of features comprising transcript-level aggregates of token-level features for tokens located within the defined distance of anchors in the entries of the plurality of entries.

42. The non-transitory computer-readable media of claim 41, wherein the transcript-level aggregates comprise at least one of a count or ratio of the token-level feature for the tokens located within the defined distance of the anchors in the entry, or an average of the token-level feature for the tokens located within the defined distance of the anchors in the entry.

43. The non-transitory computer-readable media of claim 41, wherein the set of features augments a previously-defined feature set to provide the defined feature set.

44. A networked computer system comprising:
at least one network communication subsystem;
memory; and
one or more processors configured to implement:
identifying at least one anchor comprising a class-differentiating token in the transcript;
extracting features from the speech transcript according to a defined feature set, the defined feature set including a plurality of transcript-level aggregates of token-level features for tokens located within a defined distance of each of the at least one anchor; and
providing the extracted features as input to a trained classifier model to obtain a classification.

45. The networked computer system of claim 44, wherein the at least one anchor comprises a pause in the transcript, and wherein the classification comprises a classification of the speech transcript as indicative of cognitive impairment or not indicative of cognitive impairment.

46. The networked computer system of claim 44, wherein the one or more processors is further configured to implement:
identifying the anchor in a data set comprising a plurality of entries to be classified according to a classification;
for anchors found in each entry of the plurality of entries, identifying a plurality of subsequences of different lengths, each subsequence comprising a set of tokens around the anchors;
determining which length of subsequence provides a best performing classification, the length defining a distance from an anchor, the distance comprising a number of tokens before and after the anchor; and
defining and storing a set of features comprising transcript-level aggregates of token-level features for tokens located within the defined distance of anchors in the entries of the plurality of entries.

47. The networked computer system of claim 46, wherein the transcript-level aggregates comprise at least one of a count or ratio of the token-level feature for the tokens located within the defined distance of the anchors in the entry, or an average of the token-level feature for the tokens located within the defined distance of the anchors in the entry.

48. The networked computer system of claim 46, wherein the set of features augments a previously-defined feature set to provide the defined feature set.
